(19) United States
(12) Patent Application Publication
(10) Pub. No.: US 2017/0348042 A1
(43) Pub. Date: Dec. 7, 2017

(54) CUTTING MECHANISMS FOR SURGICAL END EFFECTOR ASSEMBLIES, INSTRUMENTS, AND SYSTEMS

(71) Applicant: COVIDIEN LP, Mansfield, MA (US)

(72) Inventors: THOMAS E. DROCHNER, LONGMONT, CO (US); JAMES D. ALLEN, IV, BROOMFIELD, CO (US); GARY M. COUTURE, WARD, CO (US); DUANE E. KERR, LOVELAND, CO (US); WILLIAM H. NAU, JR., LONGMONT, CO (US); ROBERT M. SHARP, BOULDER, CO (US); DAVID J. VAN TOL, BOULDER, CO (US)

(21) Appl. No.: 15/596,050

(22) Filed: May 16, 2017

Related U.S. Application Data
(60) Provisional application No. 62/343,883, filed on Jun. 1, 2016.

(51) Int. Cl. A61B 18/14; A61B 18/12; A61B 17/00; A61B 18/00

(52) U.S. Cl. CPC A61B 18/1445; A61B 18/1206; A61B 2018/145; A61B 2018/144; A61B 2018/1226; A61B 2017/00561; A61B 2017/00557; A61B 2017/00539; A61B 2018/1455; A61B 2018/0063

(57) ABSTRACT

An end effector assembly for a surgical instrument includes first and second jaw members each including a jaw housing, an electrically-conductive tissue-treatment plate, and a longitudinally-extending channel. The first and/or second jaw member is movable relative to the other between a spaced-apart position and an approximated position. A cutting mechanism is disposed at least partially within the second jaw member. The cutting mechanism may include an inflatable bladder, a fluid line coupled to the inflatable bladder, and a knife coupled to the inflatable bladder. The cutting mechanism may alternatively include a fluid line, a knife, and a sealing member that defines a variable-volume sealed chamber within the longitudinally-extending channel of the second jaw member. The cutting mechanism may alternatively include at least one electromagnet, at least one electrical wire coupled to the at least one electromagnet, and a knife operably coupled to the at least one electromagnet.

### CROSS REFERENCE TO RELATED APPLICATION

The present application claims the benefit of and priority to U.S. Provisional Application Ser. No. 62/343,883, filed on Jun. 1, 2016, the entire contents of which are incorporated herein by reference.

### BACKGROUND

### Technical Field

The present disclosure relates to surgical devices and, more particularly, to cutting mechanisms for use with surgical end effector assemblies, instruments, and systems.

### Background of Related Art

A surgical forceps is a pliers-like instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is treated, the surgeon has to accurately sever the treated tissue. Accordingly, many electrosurgical forceps are designed to incorporate a knife or cutting member utilized to effectively sever the treated tissue.

### SUMMARY

As used herein, the term “distal” refers to the portion that is being described which is further from a user, while the term “proximal” refers to the portion that is being described which is closer to a user. Further, to the extent consistent, any or all of the aspects detailed herein may be used in conjunction with any or all of the other aspects detailed herein.

In accordance with aspects of the present disclosure, an end effector assembly for a surgical instrument is provided including first and second jaw members each including a jaw housing, an electrically-conductive tissue-treatment plate, and a longitudinally-extending channel. At least one of the first or second jaw member is movable relative to the other between a spaced-apart position and an approximated position. The end effector assembly further includes a cutting mechanism disposed within the second jaw member. The cutting mechanism includes an inflatable bladder disposed within the longitudinally-extending channel of the second jaw member, a fluid line fluidly coupled to the inflatable bladder and extending proximally from the end effector assembly, and a knife. The fluid line is adapted to connect to a source of fluid for supplying fluid to and removing fluid from the inflatable bladder to transition the inflatable bladder between a deflated condition and an inflated condition. The knife is operably coupled to the inflatable bladder and is movable between a retracted position, corresponding to the deflated condition of the inflatable bladder, wherein the knife is fully disposed within the longitudinally-extending channel of the second jaw member, and an extended position, corresponding to the inflated condition of the inflatable bladder, wherein the knife extends from the longitudinally-extending channel of the second jaw member, between the first and second jaw members, and at least partially into the longitudinally-extending channel of the first jaw member.

In an aspect of the present disclosure, the knife is mounted on the inflatable bladder.

In another aspect of the present disclosure, a guide platform is disposed within the longitudinally-extending channel of the second jaw member. In such aspects, the guide platform includes the knife mounted thereon and is configured such that, upon inflation of the inflatable bladder, the inflatable bladder urges the guide platform to urge the knife from the retracted position to the extended position.

In yet another aspect of the present disclosure, the guide platform includes a support surface having the knife disposed thereon and at least one leg extending therefrom.

In still another aspect of the present disclosure, the at least one leg is slidably disposed within at least one guide track portion of the longitudinally-extending channel of the second jaw member.

Another end effector assembly for a surgical instrument provided in accordance with aspects of the present disclosure includes first and second jaw members each including a jaw housing, an electrically-conductive tissue-treatment plate, and a longitudinally-extending channel. At least one of the first or second jaw members is movable relative to the other between a spaced-apart position and an approximated position. A cutting mechanism is disposed within the second jaw member and includes a fluid line extending proximally from the end effector assembly and adapted to connect to a source of fluid, a knife, and a sealing member engaged to the knife and sealing engaged within the longitudinally-extending channel of the second jaw member so as to define a sealed chamber therein. The fluid line is configured to supply fluid to the sealed chamber to thereby urge the knife and the sealing member from a retracted position, corresponding to a minimum-volume condition of the sealed chamber, wherein the knife is fully disposed within the longitudinally-extending channel of the second jaw member, to an extended position, corresponding to a maximum-volume condition of the sealed chamber, wherein the knife extends from the longitudinally-extending channel of the second jaw member, between the first and second jaw members, and at least partially into the longitudinally-extending channel of the first jaw member.

In an aspect of the present disclosure, the fluid line is configured to supply high-pressure pulses of fluid to the sealed chamber to move the knife from the retracted position to the extended position.

In another aspect of the present disclosure, the fluid line is configured to withdraw fluid from the sealed chamber to return the knife from the extended position to the retracted position under suction.

Another end effector assembly for a surgical instrument provided in accordance with aspects of the present disclosure includes first and second jaw members each including a jaw housing, an electrically-conductive tissue-treatment plate, and a longitudinally-extending channel. At least one of the first or second jaw members is movable relative to the other between a spaced-apart position and an approximated position. A cutting mechanism is disposed within the second jaw member and includes at least one electromagnet disposed within the jaw housing of the second jaw member, at least one electrical wire coupled to the at least one electromagnet and extending proximally from the end effector assembly to connect to a source of energy for energizing the at least one electromagnet to produce a magnetic field, and a knife at least partially disposed within the longitudinally-extending channel of the second jaw member. The knife includes a magnetic portion or has a magnetic base engaged thereto and is repelled from or attracted to the at least one electromagnet in response to magnetization of the at least one electromagnet to thereby move the knife from a retracted position to an extended position.

In an aspect of the present disclosure, in the retracted position, the knife is fully disposed within the longitudinally-extending channel of the second jaw member, and, in the extended position, the knife extends from the longitudinally-extending channel of the second jaw member, between the first and second jaw members, and at least partially into the longitudinally-extending channel of the first jaw member.

In another aspect of the present disclosure, upon magnetization of the at least one electromagnet, the knife is repelled therefrom such that the knife is urged from the retracted position to the extended position.

In yet another aspect of the preset disclosure, in the retracted position, the knife is disposed partially within the longitudinally-extending channels of the first and second jaw members at proximal ends thereof, and, in the extended position, the knife is disposed partially within the longitudinally-extending channels of the first and second jaw members at distal ends thereof.

In still another aspect of the present disclosure, the at least one electromagnet includes a series of electromagnets arranged longitudinally between the proximal and distal ends of the first and second jaw members.

In still yet another aspect of the present disclosure, the series of electromagnets are successively activated and deactivated in a proximal-to-distal direction to move the knife from the retracted position to the extended position.

In another aspect of the present disclosure, the series of electromagnets are successively activated and deactivated in a distal-to-proximal direction to return the knife from the extended position to the retracted position.

### BRIEF DESCRIPTION OF THE DRAWINGS

The above and other aspects and features of the present disclosure will become more apparent in view of the following detailed description when taken in conjunction with the accompanying drawings wherein like reference numerals identify similar or identical elements and:

FIG. 1A is a perspective view of endoscopic surgical forceps exemplifying the aspects and features of the present disclosure, wherein the shaft of the endoscopic surgical forceps is disposed in a non-articulated position and wherein the jaw members of the endoscopic surgical forceps are disposed in a spaced-apart position;

FIG. 1B is a perspective view of the endoscopic surgical forceps of FIG. 1A, wherein the shaft of the endoscopic surgical forceps is disposed in an articulated position and wherein the jaw members of the endoscopic surgical forceps are disposed in an approximated position;

FIG. 2 is a perspective view of an open surgical forceps exemplifying the aspects and features of the present disclosure;

FIG. 3 is a schematic illustration of a robotic surgical system exemplifying the aspects and features of the present disclosure;

FIG. 4A is a longitudinal, cross-sectional view of an end effector assembly configured for use with the forceps of FIG. 1A, the forceps of FIG. 2, and/or the system of FIG. 3, wherein the knife thereof is disposed in a retracted position;

FIG. 4B is a transverse, cross-sectional view of the end effector assembly of FIG. 4A, wherein the knife is disposed in the retracted position;

FIG. 5A is a longitudinal, cross-sectional view of the end effector assembly of FIGS. 4A, wherein the knife is disposed in an extended position;

FIG. 5B is a transverse, cross-sectional view of the end effector assembly of FIG. 4A, wherein the knife is disposed in the extended position;

FIG. 6 is a transverse, cross-sectional view of another end effector assembly configured for use with the forceps of FIG. 1A, the forceps of FIG. 2, and/or the system of FIG. 3, wherein the knife thereof is disposed in a retracted position;

FIG. 7 is a transverse, cross-sectional view of the end effector assembly of FIG. 6, wherein the knife is disposed in an extended position;

FIG. 8A is a longitudinal, cross-sectional view of another end effector assembly configured for use with the forceps of FIG. 1A, the forceps of FIG. 2, and/or the system of FIG. 3, wherein the knife thereof is disposed in a retracted position;

FIG. 8B is a transverse, cross-sectional view of the end effector assembly of FIG. 8A, wherein the knife is disposed in the retracted position;

FIG. 9A is a longitudinal, cross-sectional view of the end effector assembly of FIG. 8A, wherein the knife is disposed in an extended position;

FIG. 9B is a transverse, cross-sectional view of the end effector assembly of FIG. 8A, wherein the knife is disposed in the extended position;

FIG. 10A is a longitudinal, cross-sectional view of another end effector assembly configured for use with the forceps of FIG. 1A, the forceps of FIG. 2, and/or the system of FIG. 3, wherein the knife thereof is disposed in a retracted position;

FIG. 10B is a transverse, cross-sectional view of the end effector assembly of FIG. 10A, wherein the knife is disposed in the retracted position;

FIG. 11A is a longitudinal, cross-sectional view of the end effector assembly of FIG. 10A, wherein the knife is disposed in an extended position;

FIG. 11B is a transverse, cross-sectional view of the end effector assembly of FIG. 10A, wherein the knife is disposed in the extended position;

FIG. 12 is a longitudinal, cross-sectional view of another end effector assembly configured for use with the forceps of FIG. 1A, the forceps of FIG. 2, and/or the system of FIG. 3, wherein the knife thereof is disposed in a retracted position;

FIG. 13 is a longitudinal, cross-sectional view of the end effector assembly of FIG. 12, wherein the knife is disposed in an intermediate position; and

FIG. 14 is a longitudinal, cross-sectional view of the end effector assembly of FIG. 12, wherein the knife is disposed in an extended position.

### DETAILED DESCRIPTION

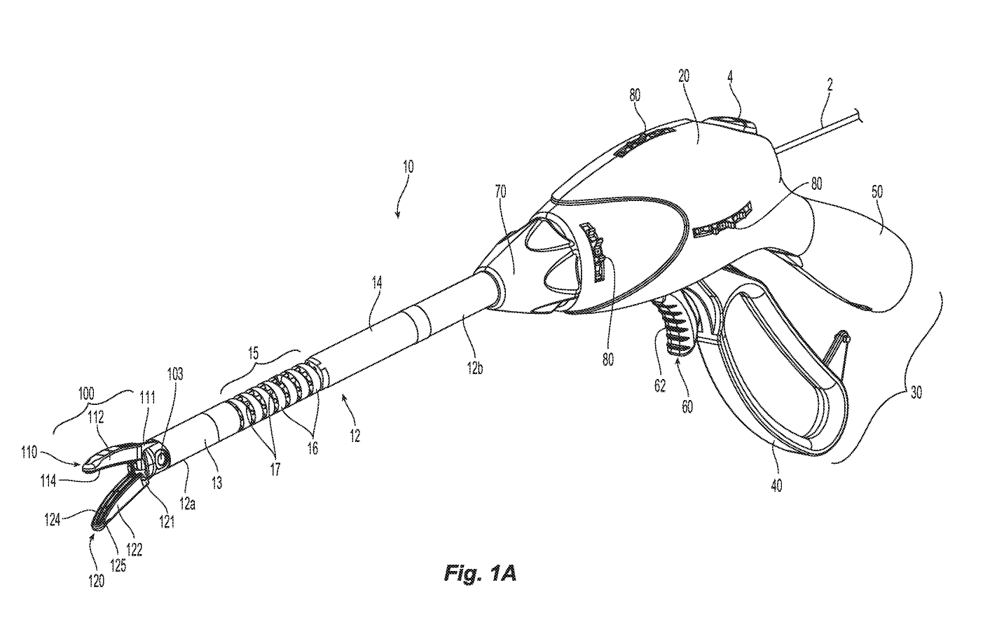

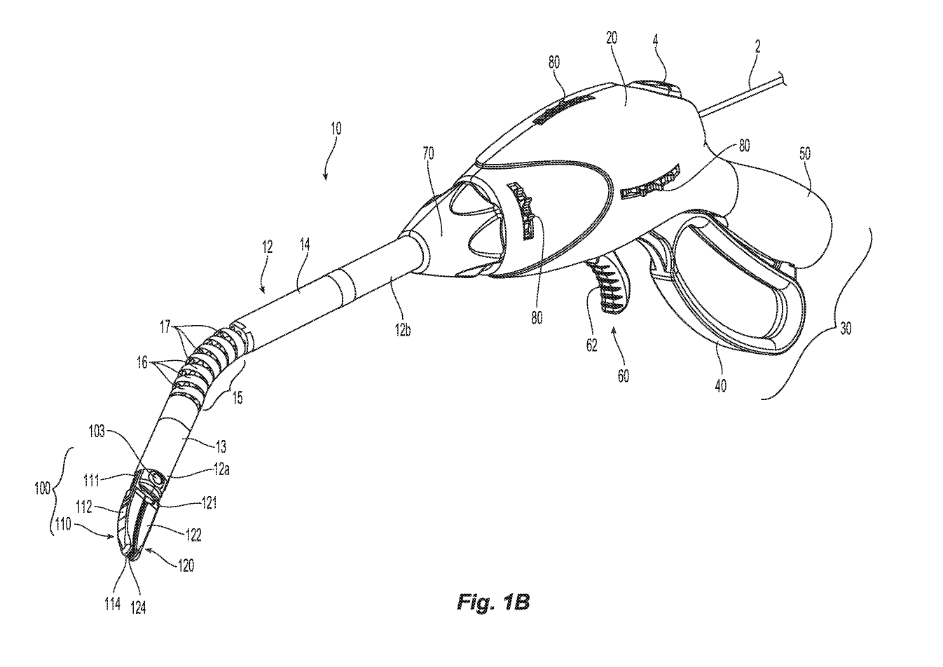

Referring generally to FIGS. 1A and 1B, an endoscopic surgical forceps exemplifying the aspects and features of the present disclosure is shown generally identified by reference numeral 10. For the purposes herein, endoscopic surgical forceps 10 is generally described. Aspects and features of endoscopic surgical forceps 10 not germane to the understanding of the present disclosure are omitted to avoid obscuring the aspects and features of the present disclosure in unnecessary detail.

Forceps 10 includes a housing 20, a handle assembly 30, a trigger assembly 60, a rotating assembly 70, a plurality of articulation actuators 80, an activation switch 4, and an end effector assembly 100. Forceps 10 further includes a shaft 12 having a distal end 12a configured to mechanically engage end effector assembly 100 and a proximal end 12b that mechanically engages housing 20. Forceps 10 also includes cable 2 that connects forceps 10 to an energy source (not shown), e.g., a generator or other suitable power source, although forceps 10 may alternatively be configured as a battery-powered device. Cable 2 includes a wire (or wires) (not shown) extending therethrough that has sufficient length to extend through shaft 12 in order to provide energy to one or both tissue-treating plates 114, 124 of jaw members 110, 120, respectively, of end effector assembly 100. Activation switch 4 is coupled to tissue-treating plates 114, 124 and the source of energy for selectively activating the supply of energy to jaw members 110, 120 for treating, e.g., cauterizing, coagulating/desiccating, and/or sealing, tissue.

Shaft 12 of forceps 10 defines a distal segment 13 positioned towards distal end 12a thereof, a proximal segment 14 positioned towards proximal end 12b thereof, and an articulating section 15 disposed between the distal and proximal segments 13, 14, respectively. Articulating section 15 includes a plurality of articulating links 16 having a plurality of articulation cables 17 extending therethrough. Each cable 17 is operably engaged at its distal end to distal segment 13 and at its proximal end to one of the articulation actuators 80 so as to enable articulation of distal segment 13 and, thus, end effector assembly 100, relative to proximal segment 14 upon actuation of one or more of articulation actuators 80. In some embodiments, articulating section 15 and articulation actuators 80 are omitted, such that shaft 12 of forceps 10 does not articulate. In either configuration, rotating assembly 70 operably couples shaft 12 to housing 20 so as to enable selective rotation of shaft 12 and, thus, end effector assembly 100, relative to housing 20.

Handle assembly 30 of forceps 10 includes a fixed handle 50 and a movable handle 40. Fixed handle 50 is integrally associated with housing 20 and handle 40 is movable relative to fixed handle 50. Movable handle 40 of handle assembly 30 is operably coupled to a drive assembly (not shown) that, together, mechanically cooperate to impart movement of one or both of jaw members 110, 120 of end effector assembly 100 about a pivot 103 between a spaced-apart position (FIG. 1A) and an approximated position (FIG. 1B) to grasp tissue between jaw members 110, 120. As shown in FIG. 1A, movable handle 40 is initially spaced-apart from fixed handle 50 and, correspondingly, jaw members 110, 120 of end effector assembly 100 are disposed in the spaced-apart position. Movable handle 40 is depressible from this initial position to a depressed position corresponding to the approximated position of jaw members 110, 120 (FIG. 1B).

Trigger assembly 60 includes a trigger 62 coupled to housing 20 and movable relative thereto between an un-actuated position and an actuated position. Trigger 62 is operably coupled to a cutting mechanism, various embodiments of which are detailed below, so as to actuate the cutting mechanism to cut tissue grasped between jaw members 110, 120 of end effector assembly 100 upon actuation of trigger 62. As an alternative to a pivoting trigger 62, a slide trigger, push-button, toggle switch, or other suitable actuator may be provided.

End effector assembly 100, as noted above, includes first and second jaw members 110, 120. Each jaw member 110, 120 includes a proximal flange portion 111, 121, an outer insulative jaw housing 112, 122 disposed about the distal portion (not explicitly shown) of each jaw member 110, 120, and a tissue-treating plate 114, 124, respectively. Proximal flange portions 111, 121 are pivotably coupled to one another about pivot 103 for moving jaw members 110, 120 between the spaced-apart and approximated positions, although other suitable mechanisms for pivoting jaw members 110, 120 relative to one another are also contemplated. The distal portions (not explicitly shown) of the jaw members 110, 120 are configured to support jaw housings 112, 122, and tissue-treating plates 114, 124, respectively, thereon.

Outer insulative jaw housings 112, 122 of jaw members 110, 120 support and retain tissue-treating plates 114, 124 on respective jaw members 110, 120 in opposed relation relative to one another. Tissue-treating plates 114, 124 are formed from an electrically conductive material, e.g., for conducting electrical energy therebetween for treating tissue, although tissue-treating plates 114, 124 may alternatively be configured to conduct any suitable energy, e.g., thermal, microwave, light, ultrasonic, etc., through tissue grasped therebetween for energy-based tissue treatment. As mentioned above, tissue-treating plates 114, 124 are coupled to activation switch 4 and the source of energy (not shown), e.g., via the wires (not shown) extending from cable 2 through forceps 10, such that energy may be selectively supplied to tissue-treating plate 114 and/or tissue-treating plate 124 and conducted therebetween and through tissue disposed between jaw members 110, 120 to treat tissue. One or both of jaw members 110, 120 may further define a longitudinally-extending channel 125 (only the channel of jaw member 120 is shown).

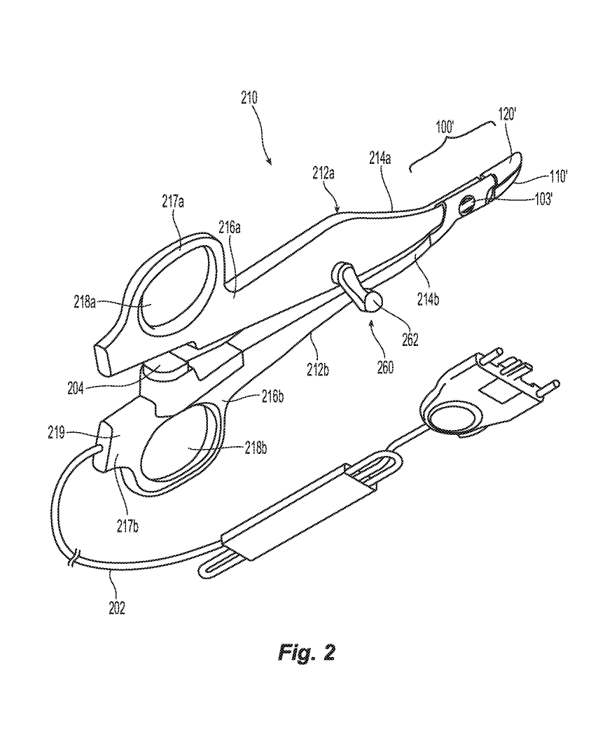

Referring to FIG. 2, an open surgical forceps exemplifying the aspects and features of the present disclosure is shown generally identified by reference numeral 210. For the purposes herein, open surgical forceps 210 is generally described. Aspects and features of open surgical forceps 210 not germane to the understanding of the present disclosure are omitted to avoid obscuring the aspects and features of the present disclosure in unnecessary detail.

Forceps 210 includes two elongated shaft members 212a, 212b, each having a proximal end 216a, 216b, and a distal end 214a, 214b, respectively. Forceps 210 is configured for use with an end effector assembly 100′ similar to end effector assembly 100 (FIGS. 1A and 1B). More specifically, end effector assembly 100′ includes first and second jaw members 110′, 120′ attached to respective distal ends 214a, 214b of shaft members 212a, 212b. Jaw members 110′, 120′ are pivotably connected about a pivot 103′. Each shaft member 212a, 212b includes a handle 217a, 217b disposed at the proximal end 216a, 216b thereof. Each handle 217a, 217b defines a finger hole 218a, 218b therethrough for receiving a finger of the user. As can be appreciated, finger holes 218a, 218b facilitate movement of the shaft members 212a, 212b relative to one another to, in turn, pivot jaw members 110′, 120′ from the spaced-apart position, wherein jaw members 110′, 120′ are disposed in spaced relation relative to one another, to the approximated position, wherein jaw members 110′, 120′ cooperate to grasp tissue therebetween.

One of the shaft members 212a, 212b of forceps 210, e.g., shaft member 212b, includes a proximal shaft connector 219 configured to connect forceps 210 to a source of energy (not shown), e.g., a generator. Proximal shaft connector 219 secures a cable 202 to forceps 210 such that the user may selectively supply energy to jaw members 110′, 120′ for treating tissue and for energy-based tissue cutting. More specifically, an activation switch 204 is provided for supplying energy to jaw members 110′, 120′ to treat tissue upon sufficient approximation of shaft members 212a, 212b, e.g., upon activation of activation switch 204 via shaft member 212a.

Forceps 210 further includes a trigger assembly 260 including a trigger 262 coupled to one of the shaft members, e.g., shaft member 212a, and movable relative thereto between an un-actuated position and an actuated position. Trigger 262 is operably coupled to a cutting mechanism, various embodiments of which are detailed below, so as to actuate the cutting mechanism to cut tissue grasped between jaw members 110,′ 120′ of end effector assembly 100′ upon movement of trigger 262 to the actuated position. Similarly as noted above, other suitable actuators for the cutting mechanism are also contemplated.

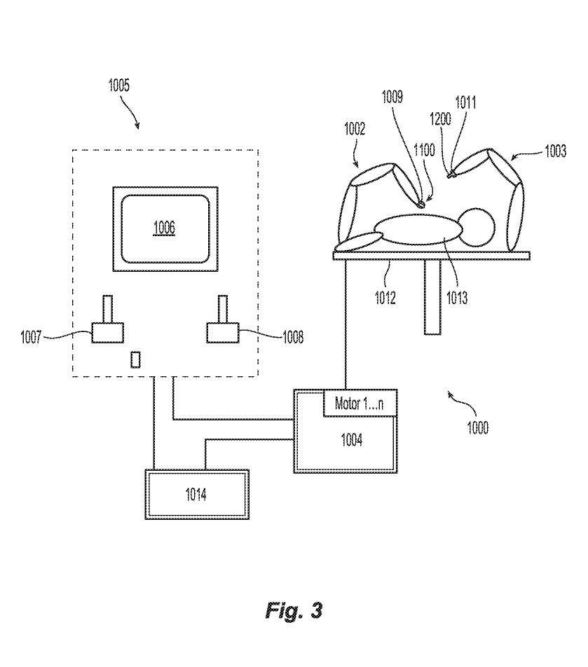

Referring generally to FIG. 3, a robotic surgical system exemplifying the aspects and features of the present disclosure is shown generally identified by reference numeral 1000. For the purposes herein, robotic surgical system 1000 is generally described. Aspects and features of robotic surgical system 1000 not germane to the understanding of the present disclosure are omitted to avoid obscuring the aspects and features of the present disclosure in unnecessary detail.

Robotic surgical system 1000 includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a surgeon may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, an end effector assembly 1100, 1200, respectively. End effector assembly 1100 is similar to end effector assemblies 100, 100′ (FIGS. 1A-1B and 2, respectively), although other suitable end effector assemblies for coupling to attaching device 1009 are also contemplated. End effector assembly 1200 may be any end effector assembly, e.g., an endoscopic camera, other surgical tool, etc. Robot arms 1002, 1003 and end effector assemblies 1100, 1200 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and end effector assemblies 1100, 1200 execute a desired movement and/or function according to a corresponding input from manual input devices 1007, 1008, respectively. Control device 1004 may also be configured in such a way that it regulates the movement of robot arms 1002, 1003 and/or of the motors.

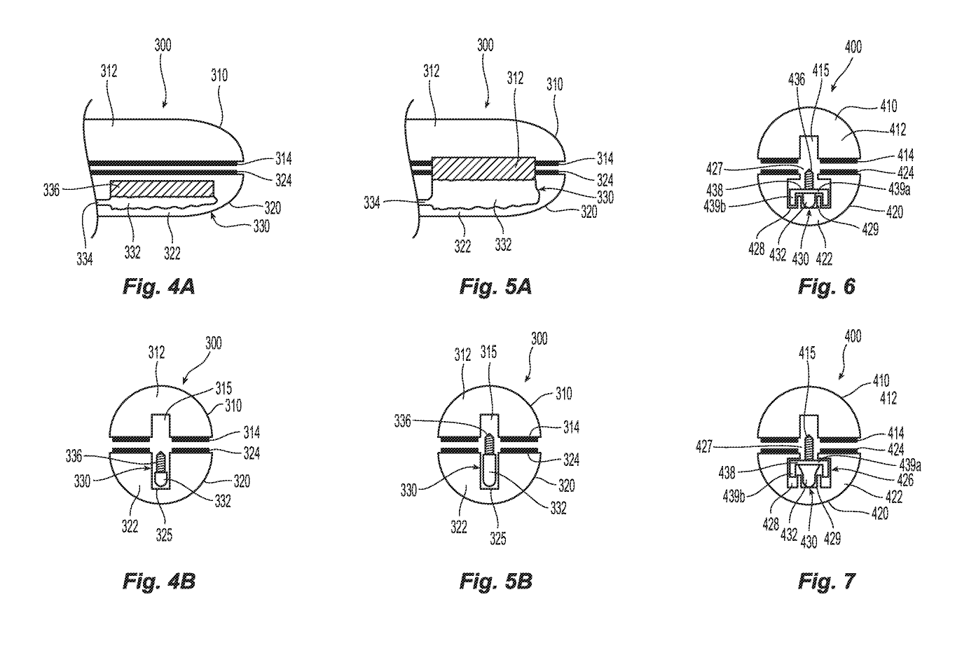

Referring generally to FIGS. 4A-14, as can be appreciated, design challenges are presented in incorporating cutting mechanisms, particularly those including elongated cutting elements, into surgical instruments having articulating shafts, e.g., forceps 10 (FIGS. 1A and 1B), open surgical instruments, e.g., forceps 210 (FIG. 2), and/or robotic surgical systems, e.g., robotic surgical system 1000 (FIG. 3). Accordingly, the various embodiments of cutting mechanisms detailed below with respect to FIGS. 4A-14 are configured to eliminate the need for elongated cutting elements, thus enabling use with articulating surgical instruments, open surgical instruments, robotic surgical systems, and any other suitable surgical instrument or system.

With reference to FIGS. 4A-5B, an end effector assembly provided in accordance with the present disclosure and configured for use with forceps 10 (FIGS. 1A-1B), forceps 210 (FIG. 2), robotic surgical system 1000 (FIG. 3), and/or any other suitable surgical instrument or system is shown generally identified by reference numeral 300.

End effector assembly 300 is similar to end effector assemblies 100, 100′, 1100 (FIGS. 1A-1B, 2, 3, respectively) and, thus, only differences therebetween will be described in detail below for purposes of brevity. End effector assembly 300 includes first and second jaw members 310, 320 each including a jaw housing 312, 322, a tissue-treating plate 314, 324, and a longitudinally-extending channel 315, 325 (FIGS. 4B and 5B).

One of the jaw members, e.g., jaw member 320, includes a cutting mechanism 330 housed within jaw housing 322 thereof. Cutting mechanism 330 includes an inflatable bladder 332 fluidly coupled to a fluid line 334 for supplying fluid to and/or removing fluid from inflatable bladder 332. Fluid, as utilized herein may refer to a liquid (e.g., water or saline), gas (e.g., air), other flowable substance, or combinations thereof. Inflatable bladder 332 is disposed within longitudinally-extending channel 325 of jaw member 320. Fluid line 334 may extend proximally from end effector assembly 300 through and/or around articulating components, pivoting components, and/or other components of the surgical instrument used with end effector assembly 300. Fluid line 334 defines a flexible configuration so as not to be interrupted by or interrupt articulation, pivoting, etc. of the surgical instrument. Fluid line 334 may ultimately be coupled to a fluid source (not shown) within the housing of the surgical instrument, an external fluid source, or other suitable fluid source. The actuator, e.g., trigger, of the surgical instrument may be operably coupled to the fluid source and/or fluid line 334 so as to supply fluid to fluid line 334 and, thus, to inflatable bladder 332, upon actuation of the trigger and to withdraw fluid from fluid line 334 and, thus, inflatable bladder 332, upon return of the trigger to an un-actuated position.

Cutting mechanism 330 further includes a knife 336 mounted on inflatable bladder 332 and disposed within longitudinally-extending channel 325 of jaw member 320. Knife 336 may define a length that extends a substantial portion of the length of jaw member 320, e.g., between 50% and 90% of the length thereof. Knife 336 is initially disposed in a retracted position (FIGS. 4A and 4B), corresponding to the deflated condition of inflatable bladder 332, wherein knife 336 is disposed within longitudinally-extending channel 325 and does not extend beyond tissue-treating plate 324. Knife 336 is deployable, upon inflation of inflatable bladder 332, from the retracted position to an extended position (FIGS. 5A and 5B), corresponding to the inflated condition of inflatable bladder 332, wherein inflatable bladder 332 urges knife 336 to extend from longitudinally-extending channel 325, between tissue-treating plates 314, 324, and at least partially into longitudinally-extending channel 315 of jaw member 310 to cut tissue grasped between jaw members 310, 320. More specifically, upon the supply of fluid via fluid line 334 to inflatable bladder 332, e.g., in response to actuation of the trigger of the surgical instrument used with end effector assembly 300, inflatable bladder 332 is expanded towards the open end of longitudinally-extending channel 325 due to inflatable bladder 332 being confined within longitudinally-extending channel 325. The expansion of inflatable bladder 332, in turn, urges knife 336 from the retracted position (FIGS. 4A and 4B) to the extended position (FIGS. 5A and 5B) to cut tissue grasped between jaw members 310, 320.

With reference to FIGS. 6 and 7, an end effector assembly provided in accordance with the present disclosure and configured for use with forceps 10 (FIGS. 1A-1B), forceps 210 (FIG. 2), robotic surgical system 1000 (FIG. 3), and/or any other suitable surgical instrument or system is shown generally identified by reference numeral 400.

End effector assembly 400 is similar to end effector assembly 300 (FIGS. 4A-5B) and, thus, only differences therebetween will be described in detail below for purposes of brevity. End effector assembly 400 includes first and second jaw members 410, 420 each including a jaw housing 412, 422, a tissue-treating plate 414, 424, and a longitudinally-extending channel 415, 425. The longitudinally-extending channel of one of the jaw members, e.g., longitudinally-extending channel 425 of jaw member 420, defines an enlarged base 426 and a narrowed opening 427. Enlarged base 426 defines a pair of outer guide tracks 428 and a central track 429 disposed between guide tracks 428.

One of the jaw members, e.g., jaw member 420, includes a cutting mechanism 430 housed within jaw housing 422 thereof. Cutting mechanism 430 includes an inflatable bladder 432 fluidly coupled to a fluid line (not shown), a knife 436, and a guide platform 438. Inflatable bladder 432 is disposed within longitudinally-extending channel 425 of jaw member 420 and, more specifically, central track 429 thereof. Guide platform 438 defines a support surface 439a and a pair of spaced-apart legs 439b extending from support surface 439a and is disposed within longitudinally-extending channel 425 of jaw member 420. More specifically, legs 439b are disposed within outer guide tracks 428 of enlarged base 426 of longitudinally-extending channel 425 so as to confine movement of guide platform 438 to towards and away from jaw member 410 in generally perpendicular orientation relative to support surface 439a.

Knife 436 is mounted on support surface 439a of guide platform 438 and is positioned to extend through narrowed opening 427 of longitudinally-extending channel 425 upon deployment of knife 436. More specifically, knife 436 is initially disposed in a retracted position (FIG. 6), corresponding to the lowered condition of guide platform 438 and the deflated condition of inflatable bladder 432, wherein knife 436 does not extend beyond tissue-treating plate 424. Upon inflation of inflatable bladder 432 and, as a result, movement of guide platform 428, knife 436 is deployed from the retracted position to an extended position (FIG. 7), corresponding to the raised condition of guide platform 438 and the inflated condition of inflatable bladder 432. That is, upon inflation of inflatable bladder 432, inflatable bladder 432 urges support surface 439a of guide platform 438 towards the raised condition which, in turn, urges knife 436 to extend through narrowed opening 427 of longitudinally-extending channel 425, between tissue-treating plates 414, 424, and at least partially into longitudinally-extending channel 415 of jaw member 410 to cut tissue grasped between jaw members 410, 420. Guide platform 438, in cooperation with enlarged base 426 of longitudinally-extending channel 425, helps ensure consistent and smooth movement of knife 436 between the retracted and extended positions (FIGS. 6 and 7, respectively) in generally perpendicular orientation relative to support surface 439a and the cutting edge of knife 436.

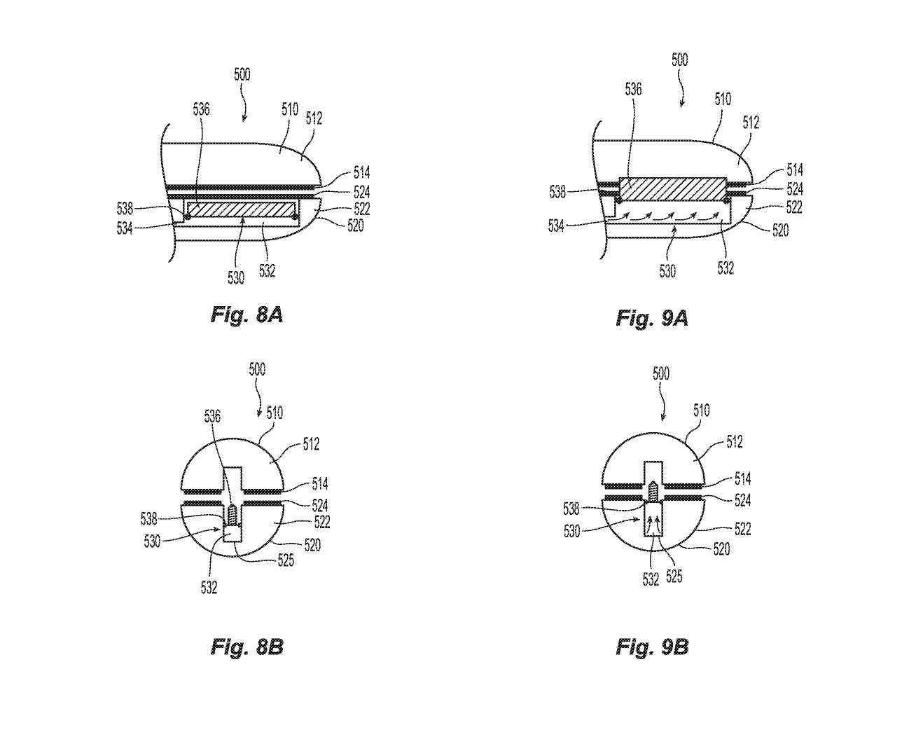

With reference to FIGS. 8A-9B, an end effector assembly provided in accordance with the present disclosure and configured for use with forceps 10 (FIGS. 1A-1B), forceps 210 (FIG. 2), robotic surgical system 1000 (FIG. 3), and/or any other suitable surgical instrument or system is shown generally identified by reference numeral 500.

End effector assembly 500 is similar to end effector assemblies 100, 100′, 1100 (FIGS. 1A-1B, 2, 3, respectively) and, thus, only differences therebetween will be described in detail below for purposes of brevity. End effector assembly 500 includes first and second jaw members 510, 520 each including a jaw housing 512, 522, a tissue-treating plate 514, 524, and a longitudinally-extending channel 515, 525 (FIGS. 8B and 9B).

One of the jaw members, e.g., jaw member 520, includes a cutting mechanism 530 housed within jaw housing 522 thereof. Cutting mechanism 530 includes a fluid line 534 for supplying fluid to and/or removing fluid from longitudinally-extending channel 525 of jaw member 520. Fluid line 534 may be configured similarly as fluid line 334 of cutting mechanism 330 (FIGS. 4A-5B). Cutting mechanism 530 further includes a knife 536 disposed within longitudinally-extending channel 525, and a sealing member 538, e.g., an elastomeric ring, gasket, or other suitable sealing member, engaged about the base of knife 536 and configured to establish a fluid-tight seal between the base of knife 536 the walls of jaw housing 522 that define longitudinally-extending channel 525 of jaw member 520. As such, a sealed chamber 532 is defined within longitudinally-extending channel 525 by the walls of jaw housing 522, sealing member 538, and knife 536. Fluid line 534 is fluidly coupled to sealed chamber 532 for supplying fluid to and removing fluid from sealed chamber 532.

Knife 536 is initially disposed in a retracted position (FIGS. 8A and 8B), corresponding to the minimum-volume condition of sealed chamber 532, wherein knife 536 is disposed within longitudinally-extending channel 525 and does not extend beyond tissue-treating plate 524. Knife 536 is deployable from the retracted position to an extended position (FIGS. 9A and 9B), corresponding to the maximum-volume condition of sealed chamber 532, wherein fluid is supplied to sealed chamber 532 to raise the pressure within sealed chamber 532 such that sealing member 538 and knife 536 are urged towards the open end of longitudinally-extending channel 525, thereby expanding the volume of sealed chamber 532. In some embodiments, the supply of fluid to sealed chamber 532 is in the form of one or more high-pressure bursts. Alternatively, a continuous supply of fluid may be provided. In either configuration, sealed chamber 532 is expanded in volume via the supply of fluid thereto and, as a result, the movement of sealing member 538 and knife 536, to the maximum-volume condition, wherein knife 536 extends from longitudinally-extending channel 525, between tissue-treating plates 514, 524, and at least partially into longitudinally-extending channel 515 of jaw member 510 to cut tissue grasped between jaw members 510, 520. Knife 536 may be returned to the retracted position, thereby returning sealed chamber 532 to the minimum-volume condition, under suction due to the withdrawal of fluid from sealed chamber 532 via fluid line 534, using a biasing member (not shown) biasing knife 536 towards the retracted position, or in any other suitable manner.

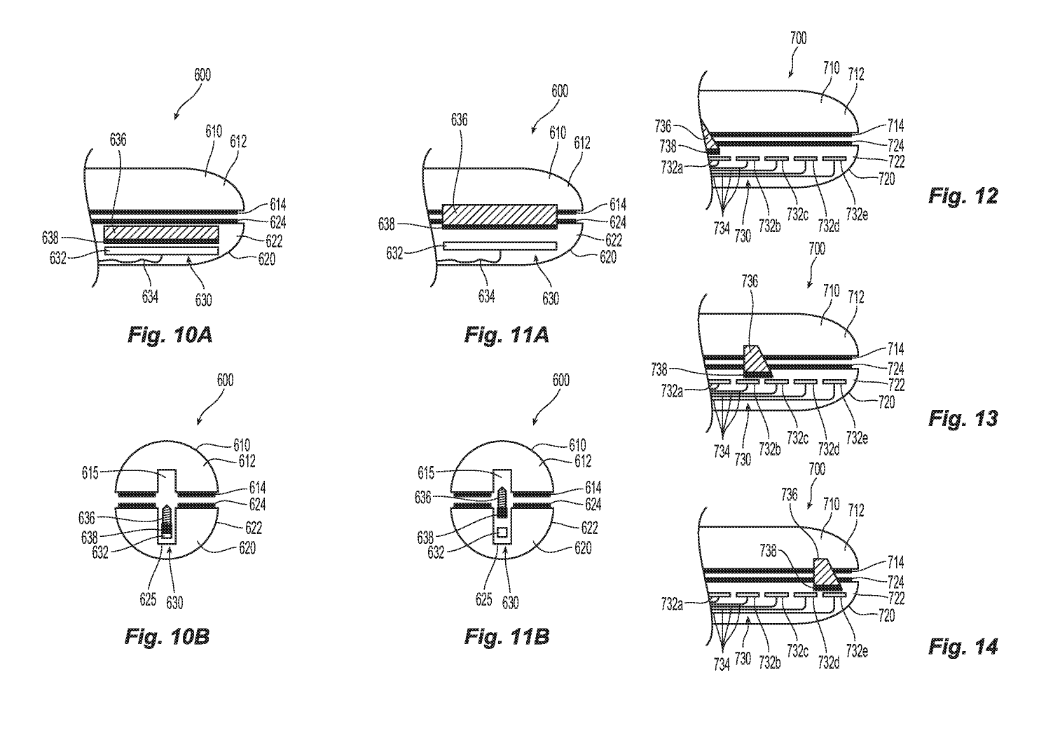

With reference to FIGS. 10A-11B, an end effector assembly provided in accordance with the present disclosure and configured for use with forceps 10 (FIGS. 1A-1B), forceps 210 (FIG. 2), robotic surgical system 1000 (FIG. 3), and/or any other suitable surgical instrument or system is shown generally identified by reference numeral 600.

End effector assembly 600 is similar to end effector assemblies 100, 100′, 1100 (FIGS. 1A-1B, 2, 3, respectively) and, thus, only differences therebetween will be described in detail below for purposes of brevity. End effector assembly 600 includes first and second jaw members 610, 620 each including a jaw housing 612, 622, a tissue-treating plate 614, 624, and a longitudinally-extending channel 615, 625 (FIGS. 10B and 11B).

One of the jaw members, e.g., jaw member 620, includes a cutting mechanism 630 housed within jaw housing 622 thereof. Cutting mechanism 630 includes an electromagnet 632 coupled to an electrical wire 634 for energizing electromagnet 632 to produce a magnetic field about electromagnet 632. Electrical wire 634 may extend proximally from end effector assembly 600 through and/or around articulating components, pivoting components, and/or other components of the surgical instrument used with end effector assembly 600. Electrical wire 634 defines a flexible configuration so as not to be interrupted by or interrupt articulation, pivoting, etc. of the surgical instrument. Electrical wire 634 may ultimately be coupled to an energy source (not shown) within the housing of the surgical instrument, an external energy source, or other suitable energy source. The actuator, e.g., trigger, of the surgical instrument may be operably coupled to the energy source and/or electrical wire 634 so as to supply energy to electrical wire 634 and, thus, to electromagnet 632, upon actuation of the trigger and to stop the supply of energy to electrical wire 634 and, thus, electromagnet 632, upon return of the trigger to an un-actuated position.

Cutting mechanism 630 further includes a knife 636 disposed within longitudinally-extending channel 625 of jaw member 620. Knife 636 may be formed from a magnetic material, or may include a base 638 engaged thereto that is formed from a magnetic material. Knife 636 may define a length that extends a substantial portion of the length of jaw member 620, e.g., between 50% and 90% of the length thereof. Knife 636 is initially disposed in a retracted position (FIGS. 10A and 10B), corresponding to the un-energized or deactivated condition of electromagnet 632, wherein knife 636 is disposed within longitudinally-extending channel 625 and does not extend beyond tissue-treating plate 624. Knife 636 is deployable, upon energization or activation of electromagnet 632, from the retracted position to an extended position (FIGS. 11A and 11B), corresponding to the energized or activated condition of electromagnet 632, wherein electromagnet 632 produces a magnet field that repels knife 636 or base 638 thereof to thereby urge knife 636 to extends from longitudinally-extending channel 625, between tissue-treating plates 614, 624, and at least partially into longitudinally-extending channel 615 of jaw member 610 to cut tissue grasped between jaw members 610, 620. Upon deactivation of electromagnet 632, knife 636 is returned to the retracted position. Return of knife 636 may be accomplished using a biasing member (not shown) biasing knife 636 towards the retracted position, via gravity, or in any other suitable fashion.

With reference to FIGS. 12-14, an end effector assembly provided in accordance with the present disclosure and configured for use with forceps 10 (FIGS. 1A-1B), forceps 210 (FIG. 2), robotic surgical system 1000 (FIG. 3), and/or any other suitable surgical instrument or system is shown generally identified by reference numeral 700.

End effector assembly 700 is similar to end effector assemblies 100, 100′, 1100 (FIGS. 1A-1B, 2, 3, respectively) and, thus, only differences therebetween will be described in detail below for purposes of brevity. End effector assembly 700 includes first and second jaw members 710, 720 each including a jaw housing 712, 722, a tissue-treating plate 714, 724, and a longitudinally-extending channel (not shown).

One of the jaw members, e.g., jaw member 720, includes a cutting mechanism 730 at least partially housed within jaw housing 722 thereof. Cutting mechanism 730 includes a series of electromagnets 732a-e (although greater or fewer than five (5) electromagnets are also contemplated) extending longitudinally along jaw member 720 from the proximal end to the distal end thereof. Cutting mechanism 730 further includes a plurality of electrical wires 734, one of which is coupled to each of the electromagnets 732a-e to provide energy thereto to produce a magnetic field about that electromagnet 732a-e. Electrical wires 734 may extend proximally from end effector assembly 700 similarly as detailed above with respect to electrical wire 634 (see FIGS. 10A and 11A) so as to couple to an energy source (not shown) and the actuator, e.g., trigger, of the surgical instrument used with end effector assembly 700.

Cutting mechanism 730 further includes a knife 736 extending between jaw members 710, 720 with a portion thereof disposed within each of the longitudinally-extending channels of jaw members 710, 720. Knife 736 may be formed from a magnetic material, or may include a base 738 engaged thereto that is formed from a magnetic material. Knife 736 is initially disposed in a retracted position (FIG. 12), corresponding to the un-energized or deactivated condition of electromagnets 732a-e, wherein knife 736 is disposed at the proximal ends of jaw members 710, 720. Knife 736 is deployable, upon successive energization or activation (and subsequent successive deactivation) of electromagnets 732a-e in the proximal-to-distal direction, from the retracted position, through an intermediate position (FIG. 13), to an extended position (FIG. 14). Such deployment of knife 736 is effected via electromagnets 732a-e being successively activated and deactivated to produce magnet fields that attract knife 736 or base 738 thereof to thereby urge knife 736 to translate longitudinally from the proximal ends of jaw members 710, 720 to the distal ends thereof to cut tissue grasped between jaw members 710, 720. Knife 736 is returned to the retracted position by successively activating (and subsequently successively deactivating) electromagnetics 732a-e in the distal-to-proximal direction, using a biasing member (not shown) biasing knife 736 towards the retracted position, or in any other suitable fashion.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An end effector assembly for a surgical instrument, comprising:
first and second jaw members each including a jaw housing, an electrically-conductive tissue-treatment plate, and a longitudinally-extending channel, at least one of the first or second jaw members movable relative to the other between a spaced-apart position and an approximated position; and
a cutting mechanism disposed within the second jaw member, the cutting mechanism including:
an inflatable bladder disposed within the longitudinally-extending channel of the second jaw member;
a fluid line fluidly coupled to the inflatable bladder and extending proximally from the end effector assembly, the fluid line adapted to connect to a source of fluid for supplying fluid to and removing fluid from the inflatable bladder to transition the inflatable bladder between a deflated condition and an inflated condition; and
a knife operably coupled to the inflatable bladder, the knife movable between a retracted position, corresponding to the deflated condition of the inflatable bladder, wherein the knife is fully disposed within the longitudinally-extending channel of the second jaw member, and an extended position, corresponding to the inflated condition of the inflatable bladder, wherein the knife extends from the longitudinally-extending channel of the second jaw member, between the first and second jaw members, and at least partially into the longitudinally-extending channel of the first jaw member.

2. The end effector assembly according to claim 1, wherein the knife is mounted on the inflatable bladder.

3. The end effector assembly according to claim 1, further comprising a guide platform disposed within the longitudinally-extending channel of the second jaw member and having the knife mounted thereon, wherein upon inflation of the inflatable bladder, the inflatable bladder urges the guide platform to urge the knife from the retracted position to the extended position.

4. The end effector assembly according to claim 3, wherein the guide platform includes a support surface having the knife disposed thereon and at least one leg extending therefrom.

5. The end effector assembly according to claim 4, wherein the at least one leg is slidably disposed within at least one guide track portion of the longitudinally-extending channel of the second jaw member.

6. An end effector assembly for a surgical instrument, comprising:
first and second jaw members each including a jaw housing, an electrically-conductive tissue-treatment plate, and a longitudinally-extending channel, at least one of the first or second jaw members movable relative to the other between a spaced-apart position and an approximated position; and
a cutting mechanism disposed within the second jaw member, the cutting mechanism including:
a fluid line extending proximally from the end effector assembly, the fluid line adapted to connect to a source of fluid;
a knife; and
a sealing member engaged to the knife and sealing engaged within the longitudinally-extending channel of the second jaw member so as to define a sealed chamber therein, wherein the fluid line is configured to supply fluid to the sealed chamber to thereby urge the knife and the sealing member from a retracted position, corresponding to a minimum-volume condition of the sealed chamber, wherein the knife is fully disposed within the longitudinally-extending channel of the second jaw member, to an extended position, corresponding to a maximum-volume condition of the sealed chamber, wherein the knife extends from the longitudinally-extending channel of the second jaw member, between the first and second jaw members, and at least partially into the longitudinally-extending channel of the first jaw member.

7. The end effector assembly according to claim 6, wherein the fluid line is configured to supply high-pressure pulses of fluid to the sealed chamber to move the knife from the retracted position to the extended position.

8. The end effector assembly according to claim 6, wherein the fluid line is configured to withdraw fluid from the sealed chamber to return the knife from the extended position to the retracted position under suction.

9. An end effector assembly for a surgical instrument, comprising:
first and second jaw members each including a jaw housing, an electrically-conductive tissue-treatment plate, and a longitudinally-extending channel, at least one of the first or second jaw members movable relative to the other between a spaced-apart position and an approximated position; and
a cutting mechanism disposed within the second jaw member, the cutting mechanism including:
at least one electromagnet disposed within the jaw housing of the second jaw member;
at least one electrical wire coupled to the at least one electromagnet and extending proximally from the end effector assembly, the at least one electrical wire adapted to connect to a source of energy for energizing the at least one electromagnet to produce a magnetic field; and
a knife at least partially disposed within the longitudinally-extending channel of the second jaw member, the knife including a magnetic portion or having a magnetic base engaged thereto, wherein the knife is repelled from or attracted to the at least one electromagnet in response to magnetization of the at least one electromagnet to thereby move the knife from a retracted position to an extended position.

10. The end effector assembly according to claim 9, wherein, in the retracted position, the knife is fully disposed within the longitudinally-extending channel of the second jaw member, and wherein, in the extended position, the knife extends from the longitudinally-extending channel of the second jaw member, between the first and second jaw members, and at least partially into the longitudinally-extending channel of the first jaw member.

11. The end effector assembly according to claim 10, wherein, upon magnetization of the at least one electromagnet, the knife is repelled therefrom such that the knife is urged from the retracted position to the extended position.

12. The end effector assembly according to claim 9, wherein, in the retracted position, the knife is disposed partially within the longitudinally-extending channels of the first and second jaw members at proximal ends thereof, and wherein, in the extended position, the knife is disposed partially within the longitudinally-extending channels of the first and second jaw members at distal ends thereof.

13. The end effector assembly according to claim 12, wherein the at least one electromagnet includes a series of electromagnets arranged longitudinally between the proximal and distal ends of the first and second jaw members.

14. The end effector assembly according to claim 13, wherein the series of electromagnets are successively activated and deactivated in a proximal-to-distal direction to move the knife from the retracted position to the extended position.

15. The end effector assembly according to claim 14, wherein the series of electromagnets are successively activated and deactivated in a distal-to-proximal direction to return the knife from the extended position to the retracted position.
